(19) United States
(12) Patent Application Publication
(10) Pub. No.: US 2022/0354750 A1
(43) Pub. Date: Nov. 10, 2022

(54) AUTOMATED ORAL COMPOUND ADMINISTRATION SYSTEM AND METHOD

(71) Applicant: IMD Solutions LLC, Tempe, AZ (US)

(72) Inventors: Zachary Smith, Tempe, AZ (US); Cade Montplaisir, Marana, AZ (US); Michael Dale Grapp, Marana, AZ (US); Mauro Robles Garcia, Wickenburg, AZ (US); Ty Promreuk, Gilbert, AZ (US)

(73) Assignee: IMD Solutions LLC

(21) Appl. No.: 17/313,790

(22) Filed: May 6, 2021

(51) Int. Cl. A61J 7/00; G16H 40/67; G16H 20/13

(52) U.S. Cl. CPC A61J 7/0076; G16H 40/67; G16H 20/13

(57) ABSTRACT

An automated oral compound delivery system configured to be removably inserted into a patient's mouth for receiving a signal from a device for intraoral delivery of a drug comprising an oral retainer that can be configured to be removably secured in the patient's mouth wherein the oral retainer can comprise at least one attachment point. An oral delivery system can comprise a housing having top plate and a bottom plate. The housing can further comprise a first storage chamber and a second storage chamber wherein a compound can be contained within the first storage chambers. An intake valve and a release valve can be attached to the housing wherein the housing is pressurized by a pressurization system that is attached to the storage chamber. A controller for activating the device and dispensing the compound into the patient's mouth.

### CROSS-REFERENCE TO RELATED APPLICATIONS

This application claims benefit from currently pending U.S. Provisional Application No. 63/023,514 titled “Methods for Oral Administration” and having a filing date of May 12, 2020, all of which is incorporated by reference herein.

### TECHNICAL FIELD

The present specification relates generally to the pharmaceutical oral dose administration devices and computerized prescription administration devices more particularly a device that receives a signal from a monitoring device and when the user is levels are lows disperses a compound into the user's mouth.

### BACKGROUND OF THE INVENTION

There exists a large number of diseases and conditions that require consistent monitoring and timely administration of medication, such as hypoglycemia, diabetes, cardiac disease, stroke, epilepsy, narcolepsy, anaphylaxis, and the like. These diseases and conditions are more easily controlled using automated drug delivery systems, such as an insulin pump. However, most automated drug delivery systems rely on intravenous drug deliver which is invasive, requires the aid of a skilled professional to insert the device, is generally difficult to and remove, and the intravenous medications are only available to a medical professional.

Implementations described herein may assist a user in receiving the aid of non-invasive automated drug delivery without the use of intravenous drugs, nor a skilled professional placing the device.

### BRIEF SUMMARY OF THE INVENTION

An automated oral compound delivery system is contemplated configured to be removably inserted into a patient's mouth for intraoral delivery of a compound. A removable oral retainer allows the patient to secure the system in the patient's mouth for an extended period. A housing is removably coupled to the oral retainer and encompasses a compound. The housing may have a top plate and a bottom plate and a first chamber and a second chamber separated by a chamber divider or may be a bladder that can change its volume as compound is expelled from the housing. The top plate and the bottom plate may be hermetically sealed to the housing. The housing may be manufactured of biocompatible materials that are formed to the patient's mouth.

A release valve selectively allows the release of the compound from the housing and ensures that the compound is not released to the patient inadvertently. The release valve may be placed in the portion of the housing most proximal to the back of the patient's throat. In a particular embodiment, the release valve is a duck bill valve. An intake valve may also be provided in an air intake hole to allow air into the housing when negative pressure in the housing builds from dispensing compound to the patient.

The housing also includes a pump to expel the compound from the housing into the patient's mouth. The compound may be housed in the first chamber and the pump may be housed in the second chamber, or the pump may reside in a single chamber with the compound. In on embodiment, the pump comprises a motor, a compressor box plate and a compressor box together provide pressure to the compound and wherein the compressor box plate is sealed against the air intake hole. The compressor box may comprise a helical rotor connected to the motor. In an alternative embodiment, the pump is selected from the group comprising a piezoelectric, electrostatic, electroactive polymer, thermo-pneumatic, shape memory alloy, thermally expandable polymer and electromagnetic.

A controller is coupled to the pump that communicates wirelessly with a remote computing device and a remote monitoring device. The controller may be coupled to and powered by a power source. The system may also include a sensor coupled to the remote monitoring device to determine the levels of an analyte in the patient.

A method of dispensing a compound orally is also contemplated. A patient is provided an oral compound delivery system to be carried in the patient's mouth for an extended period. The system continuously detects levels of an analyte related to the oral compound in a patient via a remote monitoring system and compares the patient's levels to a predetermined level that is determined for the patient. Based on the comparison, a dose is determined wherein the dose is the timing of a predetermined amount of the compound to disperse or the amount of the compound to disperse at a preset time to bring the patient's level to the predetermined level or to maintain the patient's levels at the predetermined level

The remote monitoring system sends a signal to the oral delivery system to deliver the dose and a pump is activated to deliver the dose based on the signal. In a particular embodiment, the patient's levels are glucose and blood sugar levels and the remote monitoring system is a continuous glucose monitor.

The compound may be dispersed by a pump selected from the group comprising piezoelectric, electrostatic, electroactive polymer, thermo-pneumatic, shape memory alloy, thermally expandable polymer and electromagnetic. A controller in the oral delivery system receives the signal and activates the material of the pump to expand and expel the compound from the housing.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors' intent and desire that the simple, plain and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112 (f). Thus, the use of the words “function,” “means” or “step” in the Detailed Description or Description of the Drawings or claims is not intended to somehow indicate a desire to invoke the special provisions of 35 U.S.C. § 112 (f), to define the invention. To the contrary, if the provisions of 35 U.S.C. § 112 (f) are sought to be invoked to define the inventions, the claims will specifically and expressly state the exact phrases “means for” or “step for, and will also recite the word “function” (i.e., will state “means for performing the function of [insert function]”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventors not to invoke the provisions of 35 U.S.C. § 112 (f). Moreover, even if the provisions of 35 U.S.C. § 112 (f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

Additional features and advantages of the present specification will become apparent to those skilled in the art upon consideration of the following detailed description of the illustrative embodiment exemplifying the best mode of carrying out the invention as presently perceived.

### BRIEF DESCRIPTION OF THE DRAWINGS

These and other features, aspects, and advantages of the present specification will become better understood with regard to the following description, appended claims, and accompanying drawings. In the figures, like reference numbers refer to like elements or acts throughout the figures.

FIG. 1 is a side view of an oral delivery system shown in a patient's mouth in accordance to one, or more embodiments;

FIG. 2 is a top view of an oral delivery system in accordance to one, or more embodiments;

FIG. 3 is an exploded isometric top view of an oral delivery system in accordance to one, or more embodiments;

FIG. 4 is an exploded side view of an oral delivery system in accordance to one, or more embodiments;

FIG. 5 is an exploded isometric bottom view of an oral delivery system in accordance to one, or more embodiments;

FIG. 6 is a top view of another embodiment of a oral delivery system in accordance to one, or more embodiments;

FIG. 7 is a flow diagram of communication method of a oral delivery system in accordance to one, or more embodiments;

FIG. 8 is a flow diagram of a remote device tracking a patient's levels and sending to portable computing device for an oral delivery system in accordance to one, or more embodiments; and

FIG. 9 is a flow diagram of an example embodiment of an oral delivery system delivering Glucose to a patient in accordance to one, or more embodiments; and

FIG. 10 is an isometric view of another embodiment of oral delivery system in accordance to one, or more embodiments;

Elements and acts in the figures are illustrated for simplicity and have not necessarily been rendered according to any particular sequence or embodiment.

### DETAILED DESCRIPTION

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and devices are shown or discussed more generally in order to avoid obscuring the invention. In many cases, a description of the operation is sufficient to enable one to implement the various forms of the invention, particularly when the operation is to be implemented in software. It should be noted that there are many different and alternative configurations, devices and technologies to which the disclosed inventions may be applied. The full scope of the inventions is not limited to the examples that are described below.

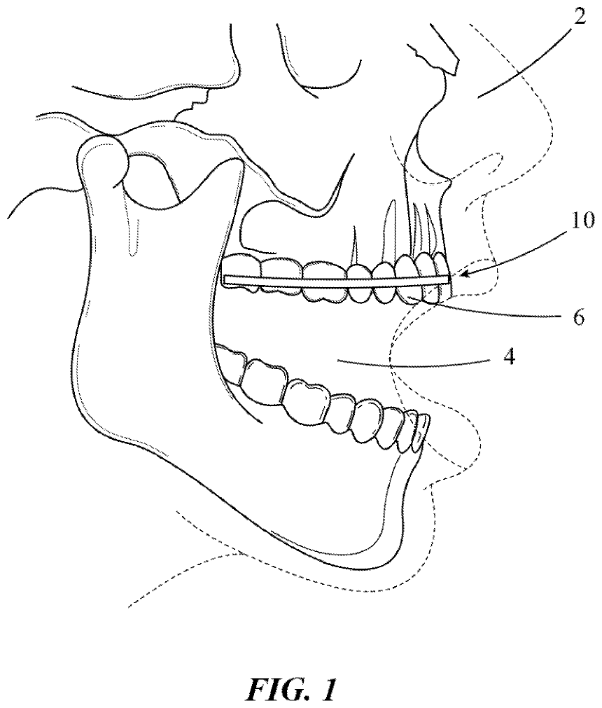

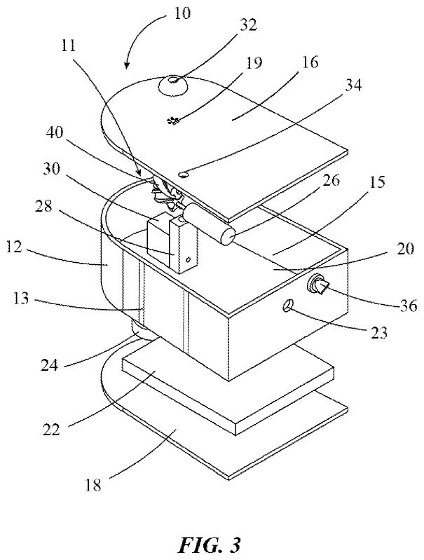

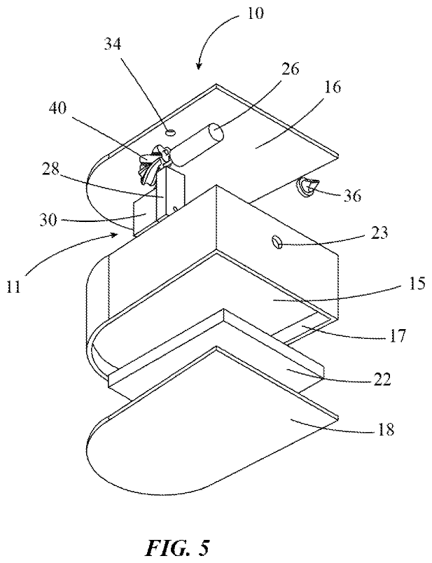

Referring initially to FIGS. 1-5, an oral delivery system 10 that can be configured to be removably inserted into a patient's mouth 4 for receiving a signal from a remote monitoring device 80 for intraoral delivery of a drug comprising a removable oral retainer 8 configured to be removably secured in the patient's mouth wherein the removable oral retainer 8 can comprise at least one attachment point 13. A dosing system 11 can be removably attached to the oral retainer by the attachment point 13 by such as, a locking groove and rail attachment, a threaded attachment, attachment by one or more pins, or any other attachment mechanism known to those having skill in the art. The oral retainer 8 can be affixed to any number of teeth 6 wherein the patient can remove the delivery system 10 when not in use. The removable oral retainer 8 can be formed and custom fit by a dentist or doctor to the patient's mouth 4. In certain embodiments the oral retainer 8 and the delivery system 10 can be incorporated into one device wherein the delivery system is incorporated into the oral retainer.

The dosing system 11 includes a controller 22, a power source 24 connected to the controller 22, a pump 25 coupled to the power source 24 and the controller 22 such that it is controlled by the controller 22 to deliver a desired amount of one or more compounds 21 through a compound interface 38 and/or a release valve 34. The power source 24 can be replaceable to allow the user to easily replace the power source with a new power source or recharge the old power source. The power source 24 can be a battery such as, a small coin cell battery, lithium-ion battery, calcium hydride, nickel chloride, alkaline battery, magnesium-ion battery or the like, and can be either replaceable or rechargeable through induction charging or USB charging. The charging component can include a haptic charging component (e.g., for charging batteries) and can be configured to charge the dosing system 25 when removed from the patient's mouth and not in use.

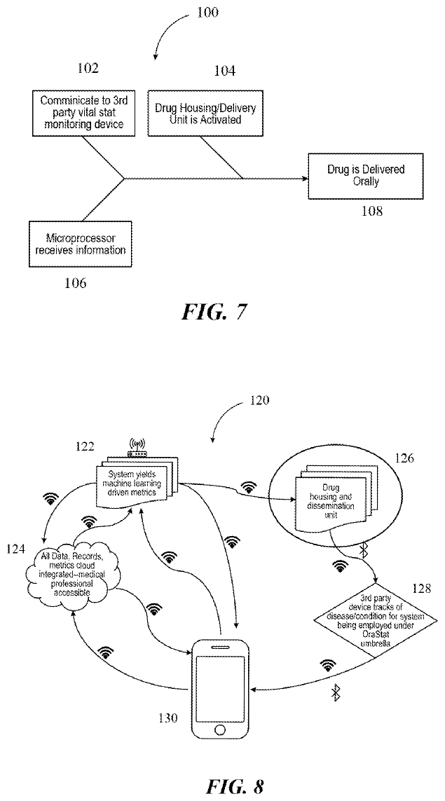

The controller 22 can communicate with a remote monitoring device 102, as shown in FIG. 7, such as a continuous glucose monitor such as Dexcom G6 or G7, Medtronic 530G pacemaker, smart device, or the like. For example, the controller 22 can be a microcontroller such as a ESP32, BLE ART, BLE UART, WC7220B, or the like that can send and receive data from the remote monitoring device and/or a remote computing system and in some embodiments the remote device and remote computing device can be a single device. The microcontroller provides remote communication by, for example, RF-radio, Bluetooth module, Bluetooth Low Energy, Wi-Fi, or the like. The remote monitoring device and/or portable computing system can wireless transmit a triggering signal to the dosing system 11 which will activate the oral delivery system and dispense the compound 21 into the patient's mouth. The portable computing device 130 as shown in FIG. 8 can be such as, a smart watch, smart phone, computer, continuous glucose monitor, or the like. In the preferred embodiment the remote monitoring device can communicate with either the controller 22 on the oral delivery system 10 or with a portable computing device 130 which can monitor and detect through an application the patient's required levels and if the patient's levels fall below the normal levels the portable computing device or remote monitoring device can send a triggering event to the controller to release the compound within the patient's mouth.

In one embodiment, the dosing system 11 is non-removably coupled to the retainer 8 at the attachment point 13. The controller 22 directs the pump 25 to take up the at least one compound 21 through a compound interface 23 and dispense the compound 21 to the patient's mouth 4. A housing 12 encompassing one or more compounds 21 is removably coupled to the compound interface 23. Alternatively, the housing 12 may be non-removably coupled to the dosing system 11 and the dosing system 11 is removably coupled to the retainer 8.

The housing 12 may be a bladder or a hard housing 12. A hard housing 12 can comprise a top plate 16 and a bottom plate 18 wherein the top plate and bottom plate can be hermetically sealed against the housing creating at least one storage chamber 14. The storage chambers 14 can be sized accordingly to fit the dosing system 11 and a compound 21 wherein the storage chambers 14 can be separated vertically in the housing or horizontally in the housing by a chamber divider 20. In the preferred embodiment, the storage chambers 14 can be separated by the chamber divider 20 horizontally creating a first chamber 15 and a second chamber 17 wherein the compound 21 can be in the first chamber 15 and the dosing system 11 can be in the second chamber 17 which can keep the dosing system 11 dry and separated away from any contaminants or the compound 21. The housing 12 can have the chamber divider 20 incorporated within it or it can be a separate part that is attached and sealed to the housing keeping the chambers separate. In certain embodiments, the chamber divider 20 can be omitted and the housing can have one storage chamber or there can be two or more chambers.

The compound 21 can be delivered out of the housing 12 into the patient's mouth 4 by a release valve 36 wherein the release valve can selectively allow the release of the compound from the housing. The chamber divider 20 can be molded into the housing 12 or can be an insert that is attached and sealed into the housing. The housing 12, top plate 16 and bottom plate 18 can be constructed from biocompatible material or polymer. In certain embodiments, the housing 12 can be a flexible substrate that can conform to the patient's mouth yet have rigidity to protect the pump 25, controller 22 and power source 24. The housing 12 can be a mixture of flexible material and a rigid substrate such as, for example, stainless steel, polypropylene, plastics, metals, or the like.

The compound 22 can be for example, a glucose solution, sucrose solution, fructose solution, concentrated carbohydrate solution, liquid carbohydrate solution, or any varying concentration and mixture of solutions in either a liquid or powdered form. In other embodiments, the compound 22 can be various form of compound or drugs that are in need of controlled dosing such as, for example, glucose, insulin, sucrose, fructose, or the like. The delivery system 10 can dispense the compound 22 at the physician's recommended dose.

The top plate 12 can comprise a first port 19 for an intake valve 32 and an air intake hole 34 wherein the top plate can be sized and shaped to cover and seal the top of the housing 12. The first port 19 can be a thru hole sized to fit the intake valve 32 wherein the intake valve 32 can be such as, a one-way valve, umbrella valve, Belleville valve, pull-in type, push-in type, or the like. The intake valve 32 can allow forward flow once the head pressure in the housing reaches an internal pressure of 0.125 psi to 10 psi wherein the head pressure can create enough force to lift the convex diaphragm of the valve from the seat which can then allow flow at the internal pressure of 0.125 psi to 10 psi wherein the valve can prevent back flow immediately in the opposite direction into or out of the housing 12. The intake valve 32 can act as a safety mechanism so that the pressure within the housing 12 does not exceed its recommended pressure. In certain embodiments, the intake valve 32 can be located on the bottom plate 18, or on either side of the housing 12.

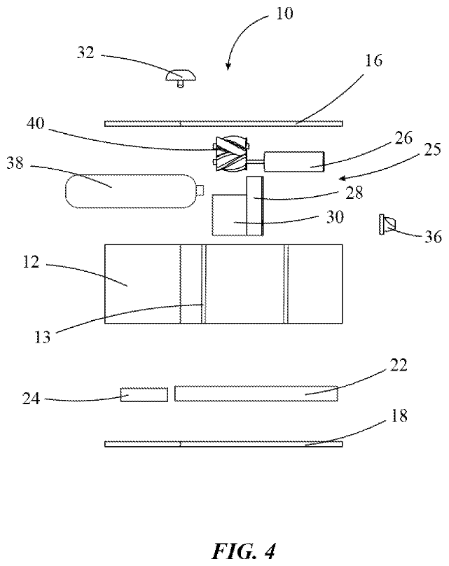

The housing 12 can comprise a second port 23 wherein the second port can be located toward the back of the housing in the direction of the patient's throat. A release valve 34 can be attached to the second port 23 wherein the release valve can be such as, duckbill valve, check valve, drip valve, cross-slit valves, dome valves or the like. The release valve 34 can allow the compound 21 to drip, dispense or spray slowly into the mouth of the user wherein the compound can be absorbed naturally with the patient's own saliva or easily swallowed. The release valve 34 can release the compound when the pressure inside of the housing 12 reaches an internal pressure of such as, for example, between 0.1 psi and 20 psi, more preferably between 0.25 psi and 10 psi, and still more preferably approximately 5 psi or the like. In certain embodiments, the second port 23 can be located near the front of the housing and the patient's mouth, or directly down onto the patient's tongue. In certain embodiments the compound 21 can be placed within a bag as shown in FIG. 4. The compound interface 38 can be placed within the first chamber and removably connected to the release valve 36 wherein the compound interface can be a bag, chamber, pouch or the like that can be made from any compressible material or any material that can be compressed and when compound 21 is release it can then return to its original shape. The pressure in the housing 12 from the pressurization system 25 can push against the bag that is filled with the compound thus dispensing the compound once the internal pressure reaches such as, for example between 0.1 psi and 20 psi, more preferably between 0.25 psi and 10 psi, and still more preferably approximately 5 psi or the like. In certain embodiments, the housing 12 can have sensors attached to the outside of it and connected to the controller 22 wherein the sensors can be such as, continuous glucose monitor, infrared sensors, oximeter, sweat sensor, saliva sensor, or the like.

The first chamber 15 can further comprise a pressurization system 25 wherein the pressurization system and/or pump 25 can comprise a motor 26 that can be attached to a compressor box plate 28 wherein the motor's shaft is attached to a compressor 30 together provide to the compound 21 and wherein the compressor box plate is sealed against the air intake hole. The compressor box plate 28 can be connected to the air intake hole 34 wherein as the motor is activated it turns the helical rotor 40 within the compressor box plate which pulls air from the patient's mouth and fills the first chamber 15 with the air thus pressurizing the first chamber. The compressor box plate 28 can be sealed against the air intake hole 34 by an O-ring, RTV silicone, gasket, rubber, or the like. The pressurizations system 25 can pressurize the housing to such as, for example, between 0.1 psi and 20 psi, more preferably between 0.25 psi and 10 psi, and still more preferably approximately 5 psi or the like. The pressurization system 25 can be attached to the first chamber 15 by such as, for example fasteners, or an attachment box within the first chamber 15. The helical rotor 30 can be manufactured from such as, for example, silicone, rubber, latex, ceramics, plastics, metals, glass-filled nylon, or the like. The helical rotor 30 can be any type of compressor, including but not limited to, compressor rotors, impellers, vanes, reciprocating or the like. In certain embodiments there can be two or more helical rotors 30 within the compressor box 30.

In another embodiment the housing 12 can further comprise an alert system (not shown) wherein the alert system can be located and attached to either the first chamber or the second chamber. The alert system can be connected to the controller 22 coupled to and powered by a power source 24 wherein when the patient's levels are low and not responding to the compound 21 the alert system can be activated alerting the user that the compound did not affect the levels and the patient will need to take another action. The alert system can be such as, a light emitting diode (“LED”), a vibrating component such as an eccentric rotating mass vibration motor, linear resonant actuator, or an auditory alarm such as a buzzer, micro-speaker, or the like.

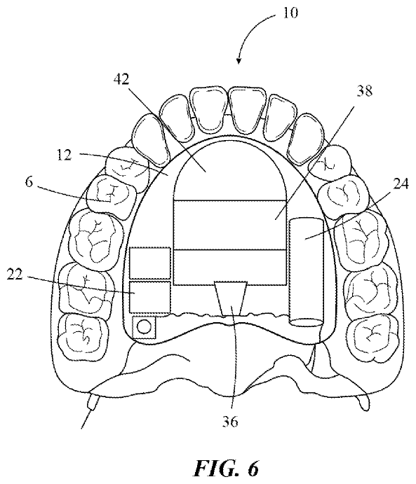

Referring to FIG. 6 showing another embodiment of the oral delivery system 10 wherein the compound bag as shown in FIG. 4 can be placed within the housing 12. The housing 12 can be pressurized by such as, nitrogen, carbon dioxide, or any other commonly used compressed gases wherein when the housing reaches a certain pressure the compound 21 can be released out of the release valve 36 into the patient's mouth. In certain embodiments the housing 12 or the first chamber 15 can be manufacture from electroactive polymer (“EAP”) or a magnetically active polymer (“MAP”) wherein when the power source applies a charge to the EAP or a magnetic field to the MAP the chamber would contract applying pressure to the compound bag 38 thus releasing the compound 21 into the patient's mouth.

Referring to FIG. 7 a flow diagram of a communication method with an oral delivery system shown generally at 100. At 102 the oral delivery system can communicate with a third party vital stat monitoring device. At 106 a controller with a microprocessor can receive the information wirelessly from a remote monitoring device which can be a trigger event wherein the trigger event can be such as low or high blood sugar or the like. At 104, the trigger event can activate the oral delivery system releasing the compound into the patient's mouth 108.

Referring to FIG. 8 a flow diagram of a remote device tracking a patient's levels and sending a trigger even to a portable computing device for an oral delivery system generally at 120. The remote monitoring device 128 can continuously track the condition or disease of the patient and that data can be collected wirelessly through a portable computing device 130 wherein the portable computing device can have the patient's set levels entered into an application wherein the application can take the data from the remote monitoring device compare it to the optimum patient's levels and then determine if a trigger event has occurred 122. If the trigger event has occurred, the portable computing device can send a signal wirelessly to the oral delivery system to dispense the compound.

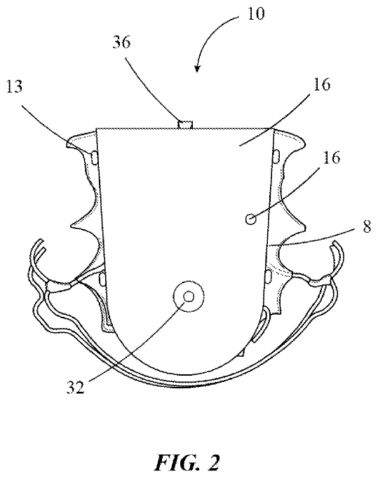

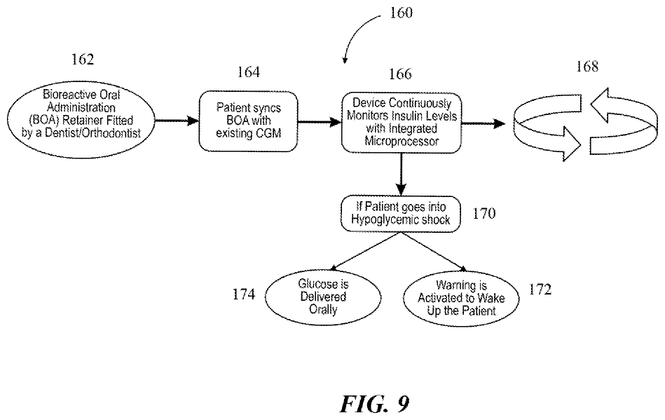

Referring to FIG. 9 a flow diagram of an example embodiment of an oral delivery system delivering glucose to a patient shown generally at 160. At 162, the patient can be fitted by a doctor with an oral retainer as shown in FIG. 2. At 164, the patient can then sync the oral delivery system with the remote monitoring system and/or a portable computing device. At 166, the remote monitoring system can continuously monitor the patient's levels such as insulin or glucose levels. At step 170, if the patient's levels are low or the patient goes into hypoglycemic shock the glucose is delivered orally to the patient by the oral delivery system at step 174. At step 172, a warning is sent to the oral delivery device to wake up the patient because their levels did not return back to normal. When a patient's levels are low the remote monitoring device can notify the portable computing device or the controller, which turn on the compressor system 25 on which can distribute the compound 21 in the back of the patient's mouth. Once the compound is released the remote monitoring device continuously monitors the patient's levels for at least five minutes monitoring if the levels stay the same, go up or start to drop. If the levels do not drop then an alarm or vibration occurs waking the patient up.

A method of dispensing a compound orally comprising providing an oral compound delivery system to a patient to be carried in the patient's mouth for an extended period. continuously detecting levels of an analyte related to the oral compound in a patient via a remote monitor. Detecting a patient's levels from a remote monitoring system, such as a patient's glucose levels sending the patient glucose levels to a portable computing device to be compared to the patient's optimum glucose levels. Comparing the patient's levels to a predetermined level that is determined by the patient and sending to the oral delivery system a trigger to turn on when the levels are low. Turning a delivery system on which turns the motor which draws air from outside of a housing into the at least one storage chamber increasing the pressure within the storage chamber. Determining a dose, wherein the dose is the timing of a predetermined amount of the compound to disperse or the amount of the compound to disperse at a preset time to be delivered to the patient to bring the patient's level to the predetermined level or to maintain the patient's levels at the predetermined level. Activating a pump to deliver the dose based on the signal and dispersing a compound through a valve into a patient's mouth. Monitoring the patient's levels after the compound has been delivered to the patient; and. Comparing the patient's levels after the compound has been administered and sending a second trigger event to the oral delivery system to sound an alarm to notify the patient. The alarm is a audible alarm or a vibration within the oral delivery system. The type of pump is selected from the group comprising piezoelectric, electrostatic, electroactive polymer, thermo-pneumatic, shape memory alloy, thermally expandable polymer and electromagnetic.

The controller will use the information from the remote monitoring system to determine if the user requires the substance or medication. In certain embodiments the controller will also comprise memory where the information from the monitor can be stored and trends can be determined. Once the controller determines that the substance or medication is needed, it signals the dispensing apparatus to release the medication. The oral delivery system then releases the medication at the rate specified by the controller. For example, a CGM may check the blood glucose every minute and send this information to an app on a cell phone. The app will record all of the information from the monitor and determine if the user's glucose is trending down. If the blood glucose is trending down, once the blood glucose level passes a threshold level, the app on the cell phone will send a signal to the dispensing apparatus to release a substrate of carbs into the oral cavity of the user. The dispensing apparatus will then release the substrate at the rate determined by the app, typically 15 ml of carbs over 5 minutes. The app on the cell phone will then record information from the monitor every 30 seconds for the next 15 minutes. At the end of 15 minutes, the app determines if the user's blood glucose is within acceptable levels, or if more intervention is required. If more intervention is required, then an alert system on the dispensing apparatus is activated. The alert system comprises any of the following: an audio alert, a visual alert, and a sensory alert. The audio alert would be noise, such as an alarm, buzzing, or siren. The visual alert would be the flashing, or constant lighting of one or more LEDs. The sensory alert would be the activation of a vibration unit, or electrical component that will shock the user. The alert system's intent is to bring the user's attention to their condition. For example, if the user is asleep, the intent is to wake them up.

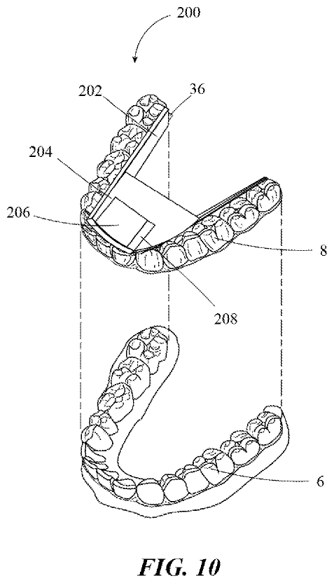

Referring to FIG. 10, an isometric view of another embodiment of an oral delivery system 200. The oral delivery system 200 can comprise an removable oral retainer 8 and a storage chamber 202 wherein the storage chamber can be attached or removably attached to the oral retainer. The oral delivery system 20 can further comprise a first chamber 204 wherein the first chamber 204 can comprise a controller 205 and a power source 208 wherein the controller 22 can communicate with a remote monitoring device 166 as shown in FIG. 9 such as a continuous glucose monitor such as Dexcom G6 or G7, Medtronic 530G pacemaker, smart device, or the like. The storage chamber 202 can further comprise a compound (not shown). The storage chamber 202 can be a bladder or a flexible pouch that can tighten and expand when an electrical charge is applied to the storage chamber. The controller 206 can receive a signal from the remote monitoring device 166 that the patient has low glucose, the controller can trigger the storage chamber 202 to contract by receiving a positive charge from the power source 208 wherein as the storage chamber contracts the compound (not shown) is dispelled from the storage chamber out of the release valve 36. The first chamber 204 can be removably attached to the storage chamber so that the user can replace the storage chamber with new compound or refill the storage chamber with new compound. The first chamber 204 can be connected to the storage chamber wherein the first chamber and the storage chamber can have electrical contacts (not shown) wherein the electrical contacts can complete the circuit allowing a charge to be carried into the storage chamber causing it contract or expand with a positive or negative charge.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present disclosure, which is defined solely by the claims. Accordingly, embodiments of the present disclosure are not limited to those precisely as shown and described.

Certain embodiments are described herein, including the best mode known to the inventors for carrying out the methods and devices described herein. Of course, variations on these described embodiments will become apparent to those of ordinary skill in the art upon reading the foregoing description. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

What is claimed is:

1. An automated oral compound delivery system configured to be removably inserted into a patient's mouth for intraoral delivery of a compound, the system comprising:
a removable oral retainer;
a housing that can be removably coupled to the oral retainer and encompasses a compound;
a pump to move the compound;
a release valve that selectively allows the release of the compound from the housing; and
a controller coupled to the pump that communicates wirelessly with a remote computing device and a remote monitor.

2. The system according to claim 1, wherein the housing has a top plate and a bottom plate.

3. The system according to claim 1, wherein the housing comprises an air intake hole.

4. The system according to claim 3, wherein the pump comprises a motor, a compressor box plate and a compressor box together provide pressure to the compound and wherein the compressor box plate is sealed against the air intake hole.

5. The system according to claim 1, wherein the pump is selected from the group comprising a piezoelectric, electrostatic, electroactive polymer, thermo-pneumatic, shape memory alloy, thermally expandable polymer and electromagnetic.

6. The system according to claim 2, wherein the top plate and bottom plate are hermetically sealed against the housing.

7. The system according to claim 4, wherein the compressor box comprises a helical rotor connected to the motor.

8. The system according to claim 1, wherein the release valve is a duck bill valve.

9. The system according to claim 1, further comprising an intake valve.

10. The system according to claim 1, wherein the release valve is located on the back of the housing in the direction of a patient's throat.

11. The system according to claim 1, wherein the housing further comprises a first chamber and a second chamber separated by a chamber divider.

12. The system according to claim 1, wherein the controller is coupled to and powered by a power source.

13. The system according to claim 1, wherein the housing is manufactured of biocompatible materials that are formed to the patient's mouth.

14. The system according to claim 1, wherein the housing is a bladder.

15. The system according to claim 1, wherein the remote computing device and the remote monitor are a single device.

16. The system according to claim 1, further comprising a sensor in communication with the remote monitoring device to measure the level of an analyte in the patient.

17. A method of dispensing a compound orally comprising:
providing an oral compound delivery system to a patient to be carried in the patient's mouth for an extended period;
continuously detecting levels of an analyte related to the oral compound in a patient via a remote monitor;
comparing the patient's levels to a predetermined level that is determined for the patient;
determining a dose, wherein the dose is the timing of a predetermined amount of the compound to disperse or the amount of the compound to disperse at a preset time to be delivered to the patient to bring the patient's level to the predetermined level or to maintain the patient's levels at the predetermined level;
sending a signal to the oral delivery system to deliver the dose;
activating a pump to deliver the dose based on the signal.

18. A method according to claim 11, wherein the patient's levels are glucose and blood sugar levels.

19. A method according to claim 11, wherein the remote monitoring system is a continuous glucose monitor.

20. A method according to claim 11, wherein the type of pump is selected from the group comprising piezoelectric, electrostatic, electroactive polymer, thermo-pneumatic, shape memory alloy, thermally expandable polymer and electromagnetic.
